(19) United States
(12) Patent Application Publication
(10) Pub. No.: US 2025/0148599 A1
(43) Pub. Date: May 8, 2025

(54) Image Overlay Method for Nerve Monitoring Systems

(71) Applicant: Viseon, Inc., Irvine, CA (US)

(72) Inventors: Peter G. Davis, Irvine, CA (US); Todd D. McIntyre, Irvine, CA (US)

(73) Assignee: Viseon, Inc., Irvine, CA (US)

(21) Appl. No.: 18/939,328

(22) Filed: Nov. 6, 2024

Related U.S. Application Data
(60) Provisional application No. 63/596,886, filed on Nov. 7, 2023.

(51) Int. Cl. G06T 7/00

(52) U.S. Cl. CPC G06T 7/0012; G06T 2207/10064; G06T 2207/30016

(57) ABSTRACT

Systems and methods provide for identification and visualization of nerves proximate a surgical site within the brain during brain surgery. The system includes a nerve monitoring system that includes a tool suitable for insertion into the brain of a patient, response sensors suitable for mounting on the skin of the patient, a nerve monitoring control system. The system also includes a fluoroscopic imaging system used to obtain fluoroscopic images of the brain and the tool during the surgery. The nerve monitoring system is interoperable with the fluoroscopic imaging system to determine the location of the tool tip corresponding a signal from the response sensors, and present a screen element in a corresponding location in the fluoroscopic images.

This application claims priority to U.S. Provisional Application 63/596,886, filed Nov. 7, 2023, pending, which is incorporated herein in its entirety.

### FIELD OF THE INVENTIONS

The inventions described below relate to the field of fluoroscopic imaging for neurosurgery.

### BACKGROUND OF THE INVENTIONS

With intra-operative fluoroscopy, video images are presented during the surgery, either as still images or video images, while the surgeon is in the operative field. That is, video images are obtained while the surgeon is manipulating tool tips within the brain, such that the viewable images include images of brain tissue, including movement of brain tissue in response to manipulation of tool tips, images of lesion tissue including movement or removal of the lesion tissue in response to manipulation of the tool tips, and images of the tools tips including movement of the tool tips as the surgeon manipulates the tool to move brain tissue and movements of the tool tips as the surgeon manipulates tools to remove the lesion tissue.

### SUMMARY OF THE INVENTIONS

The systems and methods described below provide for identification and visualization of nerves proximate a surgical site within the brain during brain surgery. The system includes a tool suitable for insertion into the brain of a patient, which may be intended to aspirate a blood mass, clip an aneurysm, cauterize a bleed in a blood vessel, remove a tumor, or merely determine the course of a nerve within the brain. The system also includes response sensors suitable for mounting on the patient's skin, in areas expected to provide signals indicative of stimulation, by applied electrical signals or by impingement, of the nerve. A nerve monitoring control system together with the tool and response sensors make up a nerve monitoring system. A fluoroscopic imaging system is used to obtain fluoroscopic images of the brain and the tool during the surgery. The nerve monitoring system is interoperable with the fluoroscopic imaging system to determine the location of the tool tip corresponding to a signal from the response sensors, and present a screen element in a corresponding location in the fluoroscopic images. With this display, a surgeon manipulating the tool tip within the brain while viewing the fluoroscopic display for guidance can avoid (or not) further close approach to the nerve, and, thus, minimize the risk of injury to the nerve. A similar system which provides for detection of a tool tip location and fluorescence detection, and addition of corresponding screen elements indication locations within the brain which fluoresce, to help identify small masses of cancerous tissue.

### BRIEF DESCRIPTION OF THE DRAWINGS

FIG. 1 illustrates the head of a patient with an area requiring surgical intervention.

FIG. 2 shows a displayed image of the patient and a displayed image of the tool along with a screen element corresponding to tool tip locations which lead to stimulation of a nerve.

FIG. 3 shows a displayed image of the patient and a displayed image of the tool along with a screen element corresponding to a course of a nerve.

FIG. 4 illustrates a subsequent insertion or manipulation of a tool, after the system has added the screen element to the image.

### DETAILED DESCRIPTION

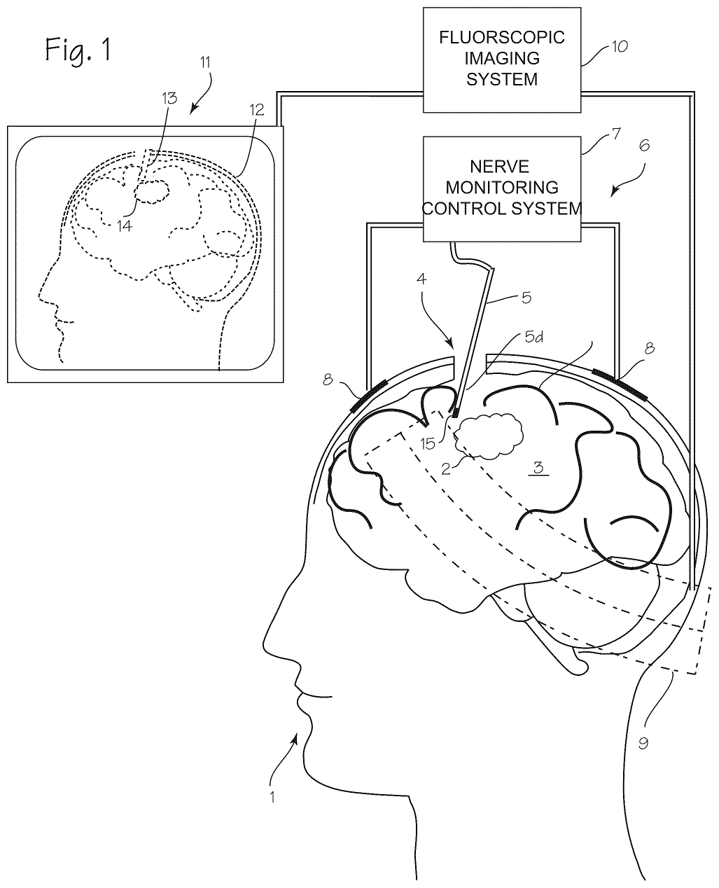

FIG. 1 illustrates a patient 1 with a mass 2 in the brain 3 that necessitates surgical intervention. The mass may be a blood mass, tumor, abscess, AVM, or foreign object (a previously placed electrode). A surgeon has created an opening through the patient's skull, with a burr hole or craniotomy 4. This figure depicts insertion of an intracranial tool 5 (a retractor) into the brain, being used to push folds of the brain aside to provide access to the mass 2. FIG. 1 also illustrates a nerve monitoring system 6, including the intracranial tool 5, a nerve monitoring control system 7, and one or more response sensors 8 disposed at locations on the patient's body, and a fluoroscopic imaging system including an imager 9 (an X-ray collimator and camera, on opposite sides of the head), control system/image processor 10 and fluoroscopic monitor display 11. The imaging system is operable to obtain images of the patient, and present an image 12 of the patient's brain, including an image 13 of the tool tip 5d within the brain and a fluoroscopic image 14 of the mass.

Depending on the nerve monitoring system mode used, the tool may comprise an electrode 15 for passing electrical pulses into nearby tissue, and response sensor 8 (monitoring electrodes in some systems) disposed on the body (in this case, the patient's scalp or face). If the nerve monitoring system is operated to apply electrical stimulation to a nerve to be located, the response sensor(s) will detect nerve signals corresponding to the applied electrical stimulation, indicating that the tool is near a nerve that corresponds to the site on the body to which the response sensors are attached. If the nerve monitor system is operated without application of electrical stimulation, the response sensor may detect nerve signals arising from impingement of the tool upon the nerve to be located (or tissue near enough to the nerve such that impingement on the near tissue stimulates the nerve). Thus, a tool may be operable to stimulate nerves proximate the tool either actively, with transmission of stimulating pulses, or passively, through impingement on the nerve or nearby tissue without the application of stimulating pulses.

The intracranial tool may be a retractor, aspirator, a macerator or other tool. The tool may include an electrode for passing electrical pulses into nearby tissue, and comprise part of a system with an electrical signal source for generating electrical signals and transmitting those signals into the brain through the tool. The tool may be a simple rod with no functionality other than retraction or mere insertion into tissue. The intracranial tool may be a probe specifically configured for manipulation within the brain to locate the nerves, in conjunction with the remainder of the nerve monitoring system.

The response sensors are operable to (1) detect nerve impulses which are caused by the electrical stimulation from the electrodes or (2) detect nerve impulses which are caused by the impingement (or other interaction) of the tool upon a nerve. They are placed about the body at locations at which stimulation (electrical or mechanical) of a nerve near the mass or the surgical pathway leading to the mass will result in a detectable signal.

While manipulating the tool, with the tip of the tool in the brain, the surgeon will be operating the intraoperative imaging system to obtain video images of the operative field (the brain, tool and mass). That is, the surgeon need not suspend manipulation of the tool or work on the brain and/or mass. At the same time, the surgeon will operate the nerve monitoring system which will detect when the distal portion of the tool is near a nerve, such that the nerve monitoring system will provide a signal indicative of proximity of the tool to the nerve to the fluoroscopic imaging system, or a human-sensible indication (visual, including indicia (signs, distinguishing images, text, or alteration in the displayed image of the tool) or audio or haptic, with appropriate displays on the tool or a display, annunciators associated with the system or on the tool, or haptic feedback devices associated with the system or on the tool). The nerve monitoring system, or a separate control system, or an image processing system of the fluoroscopic imaging system, may, operating according to software controlling the nerve monitoring system, identify the fluoroscopic image of the tool tip (1) upon identification of the fluoroscopic image of the tool tip and correlation of the image to the signal indicative of proximity of the tool to the nerve, performed by the image processing system without further input from an operator, track that position of the tool tip and thereafter operate to present a screen element on the display corresponding to that position or (2) upon automated identification of the fluoroscopic image of the tool tip or operator input, identify the image of the tool tip followed by operator input to one of the systems, indicating that the image of the tool tip is correlated to the signal indicative of proximity of the tool to the nerve and track that position of the tool tip and thereafter operate to present a screen element on the display corresponding to that position. The tip portion location is thereby correlated to an excitation or stimulation of the nerve, which may occur upon electrical stimulation of the nerve or physical impinge of the tool tip on the nerve or nearby tissue, recorded by the system, and represented on the display by a screen element. Multiple instances of stimulation, detection, recording and addition of screen elements may be accomplished as the surgeon manipulates the tool to locate the tool tip in different locations in the surgical site within the brain. Manipulation of the tool may be (1) primarily intended for treatment while nerve monitoring is incidental to the manipulation of the tool for treatment or (2) primarily intended to map the location of a nerve, with treatment to follow with a separate tool.

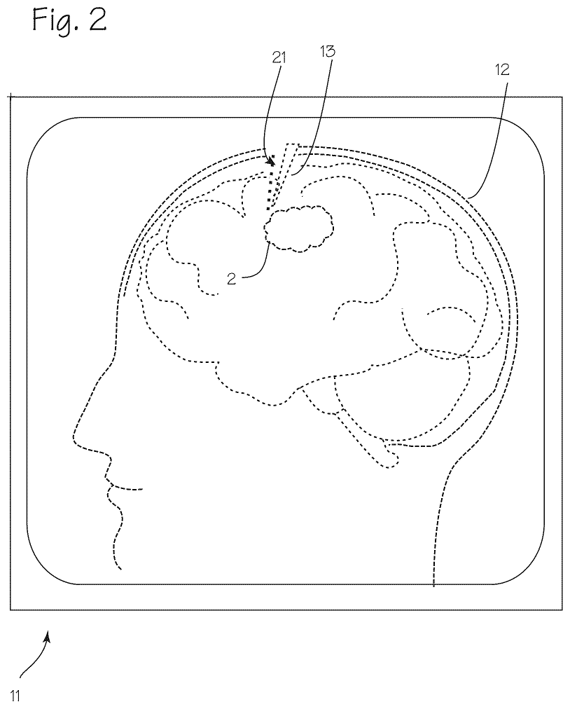

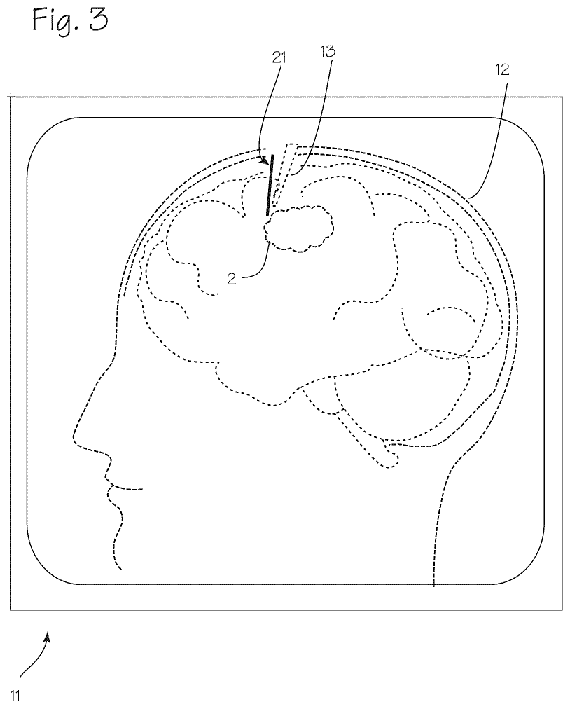

FIG. 2 shows a displayed image of the patient and a displayed image of the tool. This is preferably a video image, but a static image may be used. The image includes a fluoroscopic image 12 of the patient's brain, the image 13 of the tool tip and the image 2 of the mass. In addition, the image processing system of the fluoroscopic imaging system has added a screen element 21 to the display. The screen element 21 indicates a position or series of positions of the tool tip obtained contemporaneously with an indication from the nerve monitoring system that a nerve of interest has been stimulated (based on a signal obtained from a response sensor). In the illustration, the screen element is a series of points, with each point presented on the display in a location matching a location of the image of the tip at a time when the sensor detects stimulation due the tool tip. The screen element may be a series of images that replaces other image pixels of the fluoroscopic image so as to appear as a track of images superimposed or overlain on the fluoroscopic image, or any image visually discernable from the remainder of the display. The screen element may be added in response to user input, where a surgeon or technician provides input to the fluoroscopic imaging system to present the screen element, in response to an indication from the nerve monitoring system that the tool has stimulated a nerve. As shown in FIG. 3, the screen element can comprise a line, rather than a track of elements shown in FIG. 2.

The screen element may be added in response to input from the nerve monitoring system, input to the fluoroscopic imaging system, where the nerve monitoring system provides input to the fluoroscopic imaging system to present the screen element, in response to an indication from the response sensor that the tool has stimulated a nerve, in which case the imaging processing system of the fluoroscopic imaging system may be operable, with appropriate programming, to identify the image of the tool within the overall fluoroscopic image and generate and display the screen element in a position corresponding to the position of the tool when the response sensor indicates that the nerve has been stimulated by the tool.

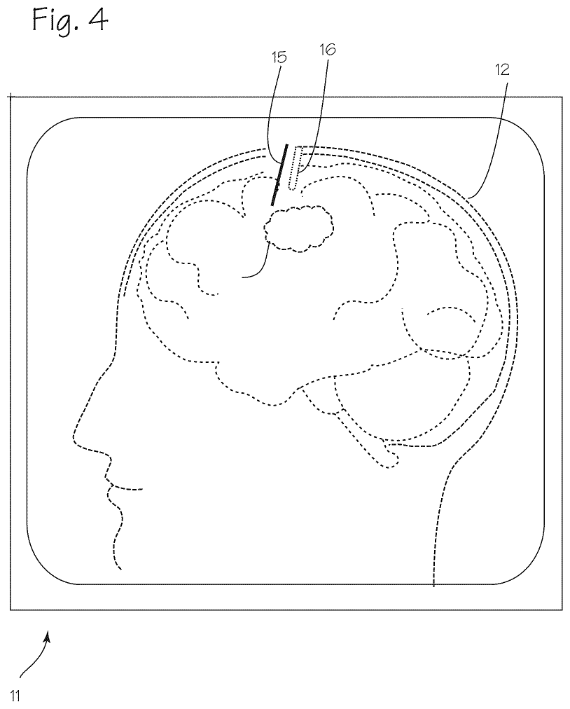

FIG. 4 illustrates a subsequent insertion or manipulation of a tool, after the system has added the screen element to the image. The image also includes the tool 5 or another tool 22 (distinct from the tool used to map the points at which stimulation of the nerve is detected) as it is being manipulated by the surgeon. Using the fluoroscopic image and screen element as a guide, the surgeon can insert the tool while observing the fluoroscopic image, and maintain some distance between the screen element and the fluoroscopic image of the tool (and thus maintain some distance between the actual nerve and the actual tool), so as to avoid stimulating or impinging upon the nerve, and thereby avoid damage to the nerve.

In use, the system may be used to perform a method of visualizing a surgical site within the brain of a patient, including displaying an image of a tool within the surgical site, identifying and keeping track of nerves within the brain.

The system may also be used to perform a method of visualizing a surgical site within the brain of a patient, including displaying an image of a tool within the surgical site, identifying and keeping track of tool tip locations within the brain at which the system detected a response by a nerve to the presence of the tool (whether due to electrical stimulation from the probe or due to presence of the probe tip at that location).

The method may also the step of obtaining a fluoroscopic image of the brain while manipulating tools within the brain and operating an image processing system to display a fluoroscopic image of the brain while manipulating tools while also simultaneously operating a nerve monitoring system to detect stimulation of a nerve caused by the tool. The method also entails, upon detecting stimulation of the nerve caused by the tool, causing an image processing system to display a screen element in a position of the displayed fluoroscopic image corresponding to the position of the tool tip when stimulation is detected. If the tool tip locations upon detection are expected to correspond very closely to the location and/or course of a nerve of concern, the scree element may be considered as the actual position of a nerve. If the tool tip locations which correspond to stimulation detections are expected to loosely correspond to the track of a location and/or course of a nerve of concern (as may be the case with electrical stimulation which may cause stimulation of a nerve in a volume of tissue and not necessarily limited to a point at which the tool tip touches a nerve), the screen element may be taken merely as an area proximate a nerve.

With the screen element as a guide, an operator can subsequently introduce and manipulate tools, using the screen element and contemporaneous fluoroscopic images of the tools tip to guide the tool tip. Typically, the operator will manipulate the tool tips to perform operations within the brain, while avoiding the area corresponding to the screen element, but may also use the screen element as a guide to manipulating the tool tips toward the location of the detected nerve.

The control system/image processor 10 may comprise a digital or analog image signal processor or it may comprise a microcontroller, a general-purpose computer, or the controller may comprise a special purpose computer, or similar device which comprises a processor and memory including program code with the memory, where the computer program code is configured with the processor to cause the system to perform the functions described throughout this specification, such as identifying the fluoroscopic image or image data of the tool and generating a corresponding screen element image and causing the display screen to display the screen element with the fluoroscopic image of the brain.

The method of detection, correlation of the tool tip with a location in the fluoroscopic image and addition of a screen element to the fluoroscopic image may be employed to identify, or confirm the absence of, fluorescing cancerous tissue within the brain. For this method, the tool includes an excitation light source (blue light, UV light, etc.) which may be disposed on the distal tip of the tool or zz and an emitted light sensor (operable to detect emitted fluorescing light, which may be green, red, infrared, etc.) disposed on the distal end of the tool. The remaining components of the system are similar to those described above. As with the nerve monitoring method, the system is operable to detect emitted light deep within brain tissue and track that position of the tool tip and thereafter operate to present a screen element on the display corresponding to that position. This method entails, upon detecting emitted fluorescing light, causing the image processing system to display a screen element in a position of the displayed fluoroscopic image corresponding to the position of the tool tip when emitted fluorescing light is detected. In use, a surgeon may, before, during or after use of a tool to remove cancerous cells, use a tool (the same tool or another) with the emitter and detector by inserting it into the brain, in a cancerous mass and tissue proximate the cancerous mass, to locate small cancerous masses or confirm the absence of such small cancerous masses proximate any more readily visualizable masses seen on the fluoroscopic image. This allows for intraoperative identification of infiltrated regions of glioma which may escape visualization in pre-operative imaging and intraoperative fluoroscopy. (This method requires administration of a fluorescence agent such as heptamethine dye or a fluorescence precursor such as 5-ALA, to the patient.)

The figures illustrate the system and method with sagittal cross-sectional views (side views) of the brain. The system can also be implemented using a coronal cross section (front-on) or transverse cross section (top view), and also with 3-D images of the brain when used with imaging systems capable of obtaining 3-D images or reconstructing 3-D images of the brain.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

We claim:

1. A system for visualizing a surgical site within the brain of a patient, including identifying and keeping track of nerves within the brain, said system comprising:
a nerve monitoring system comprising a tool operable to stimulate a nerve within the brain, a response sensor operable to detect stimulation of the nerve, and a nerve monitoring control system for receiving signals from the response sensor indicative of stimulation of the nerve by the tool and providing a signal to an operator or to the fluoroscopic image processing system;
an image processing system and display screen, wherein said image processing system is operable to generate fluoroscopic images and display fluoroscopic images on said display screen, including a fluoroscopic image of the tool within the brain;
the image processing system being interoperable with the nerve monitoring system to correlate the fluoroscopic image of the tool with a signal from the response sensor indicative of stimulation of the nerve by the tool;
wherein the image processing system is configured to generate a screen element corresponding to the location of the image of the tool on the display screen when input from the nerve monitoring system or input from an operator indicates that the tool has stimulated the nerve and display said screen element on the display screen at a location on the display screen corresponding to the location of the image of the tool.

2. The system of claim 1, wherein the image processing system is operable to identify the fluoroscopic image of the tool and generate and display the screen element at a position on the fluoroscopic image of the brain, said position corresponding to the position of the tool within the image of the brain upon receiving input that the nerve monitoring system, or an operator, provides input indicating the tool has stimulated the nerve.

3. A method of visualizing a surgical site within the brain of a patient, including identifying and keeping track of nerves within the brain, said method comprising the steps of:
obtaining a fluoroscopic image of the brain while manipulating a tool within the brain and operating an image processing system to display a fluoroscopic image of the brain while manipulating the tool;
operating a nerve monitoring system to detect stimulation of a nerve caused by the tool;
causing an image processing system to display a screen element in a position of the displayed fluoroscopic image corresponding to the position of the tool upon detecting stimulation of the nerve caused by the tool.
